(19) United States
(12) Patent Application Publication
(10) Pub. No.: US 2023/0022521 A1
(43) Pub. Date: Jan. 26, 2023

(54) NITRIC OXIDE NASAL DELIVERY APPARATUS AND METHODS

(71) Applicant: SYK Technologies LLC, Newport Beach, CA (US)

(72) Inventors: C. Michael Miller, Anaconda, MT (US); David A. Bell, Farmington, UT (US)

(21) Appl. No.: 17/814,092

(22) Filed: Jul. 21, 2022

Related U.S. Application Data
(60) Provisional application No. 63/224,552, filed on Jul. 22, 2021.
(60) Provisional application No. 63/237,337, filed on Aug. 26, 2021.

(51) Int. Cl. A61K 33/00; A61P 3/10; A61M 16/12; A61M 15/00

(52) U.S. Cl. CPC A61K 33/00; A61P 3/10; A61M 16/12; A61M 15/0025; A61M 2205/0216; A61M 2202/0275

(57) ABSTRACT

Systems, products, compounds and methods can be applied and/or used by a person to facilitate and promote the production of nitric oxide to be used by a person for health benefits. The systems, products, and devices include a nitric oxide nasal delivery system. The system can comprise one or more containers for compounds that when mixed are adapted to produce nitric oxide gas, at least one container with a nitrite compound and at least one container with an acid compound. Containers can be ruptured so that the compounds may be mixed and produce nitric oxide gas. The nitric oxide gas may then be delivered to a user via one or more of the nasal cavities and/or mouth.

### RELATED APPLICATIONS

This patent application claims the benefit of U.S. Provisional Patent Application Ser. No. 63/224,552, filed Jul. 22, 2021, and U.S. Provisional Patent Application Ser. No. 63/237,337, filed Aug. 26, 2021, which are hereby incorporated by reference in their entireties. Any and all applications for which a foreign or domestic priority claim is identified in the Application Data Sheet as filed with the present application are hereby incorporated by reference under 37 CFR 1.57.

### BACKGROUND

### Field of the Invention

The present disclosure relates to systems, products, apparatus and methods for nasal delivery of nitric oxide gas, and more specifically for devices and configurations for single-use, disposable, and/or reusable production of nitric oxide gas for delivery via a user's nasal passages or mouth.

### Description of the Related Art

There are numerous benefits documented regarding the use of nitric oxide to help or treat humans having a variety of ailments, diseases, illnesses, or afflictions. The discovery of certain nitric oxide effects in live tissue garnered a Nobel Prize. Much of the work in determining the mechanisms for implementing, and the effects of, nitric oxide administration are reported in literature.

Nitric oxide is considered an unstable molecule. This makes it difficult to develop a product that can consistently promote the formation of, or provide usable, nitric oxide to a person.

### SUMMARY OF THE INVENTION

What is needed are systems, products, apparatus, and methods of application that are easy to use, portable, reusable, and/or disposable that can also deliver a dose of nitric oxide gas to a user via the user's nasal cavities and/or mouth. Some embodiments of products, compounds, systems, and methods in accordance with this disclosure provide a nitric oxide gas product and systems adapted for nasal application that can be applied or used by a person, especially with respect to the treatment of various maladies, illnesses, biofilms, injuries, or the like. The product and systems may be suitably formulated to promote production of nitric oxide for use in human cells and tissues.

According to some embodiments, systems, methods, products, and/or devices two containers can be provided with compounds suitable for producing nitric oxide gas. For example, at least one container can comprise a nitrite compound and at least one container can comprise an acid compound. The system may be adapted such that the compounds can be mixed. For example, according to one embodiment, one or more of the containers can be configured to be easily rupturable by a user so that the compounds may be mixed and produce nitric oxide gas. Other systems for separating and then combining compounds to produce an effective gas treatment are also contemplated and are described in more detail herein. According to some embodiments, nitric oxide gas is delivered to a user via the user's nasal cavities and/or mouth. According to some embodiments one or more filters are provided. According to some embodiments, one or more nozzles are provided. According to some embodiments, a rupture component is utilized to rupture one or more containers as desired. According to some embodiments, one or more solutions and/or reactants are mixed in an inert environment. According to some embodiments, an inert environment can comprise one or more of nitrogen gas and/or carbon dioxide gas.

According to some embodiments, a nitric oxide nasal delivery system comprises a container housing a nitrite component and an acidic component adapted to generate nitric oxide gas when combined. The nitrite component and the acidic component are configured to be separated by at least one divider during a storage period prior to generation of nitric oxide gas. The nitrite component and the acidic component are configured to be selectively combined by a user during a gas generation period to generate nitric oxide gas within the container. The selective combination is activated by the user selectively compromising the at least one divider separating the nitrite component and the acidic component, to enable mixing of the nitrite component and the acidic component within a reaction zone within the container, and to initiate the gas generation period. A cap is configured to be removably coupled to the container and adapted to seal the container during the storage period and during at least a portion of the gas generation period when coupled to the container. The cap is adapted to be removed from the container during a nasal delivery period when nitric oxide gas is adapted to be delivered from the reaction zone within the container, through an exit of the container. At least a portion of the container can form a nozzle portion in some embodiments. At least a portion of the cap can form a nozzle portion in some embodiments. A pump can be provided in some embodiments. A gas filter and/or membrane filter is positioned between a reaction zone of the container and an exit of the container and/or an exit of a nozzle or cap. The gas filter and/or membrane filter is configured to allow at least one dose of nitric oxide gas to pass through the gas filter and/or membrane filter. The gas filter and/or membrane filter is configured to limit nitrogen dioxide gas from passing through the gas filter and/or membrane filter by substantially trapping nitrogen dioxide gas within the reaction zone of the container during the nasal delivery period. A droplet filter and/or vapor filter and/or mist eliminator is positioned between the reaction zone of the container and the exit of the container and/or an exit of a nozzle or cap. The droplet filter and/or vapor filter is configured to allow at least one dose of nitric oxide gas to pass through the droplet filter and/or vapor filter. The droplet filter and/or vapor filter is configured to limit droplets and/or mist or water from passing through the droplet filter and/or vapor filter by substantially trapping droplets, vapor, and/or mist within the reaction zone of the container during the nasal delivery period. The system is adapted to safely store separately the nitrite component and the acidic component during the storage period. The system is adapted to safely generate at least one dose of nitric oxide gas during the gas generation period after the divider is selectively compromised by the user. The system is adapted to safely deliver at least one dose of nitric oxide to the nasal cavity and/or mouth of the user during the nasal delivery period.

According to some embodiments, the container comprises a body portion that is compressible. According to some embodiments, the nitrite component comprises one or more of potassium nitrite and sodium nitrite. According to some embodiments, the acidic component comprises one or more of hydrochloric acid, ascorbic acid, salicylic acid, and citric acid. According to some embodiments, the divider comprises at least one impermeable membrane. According to some embodiments, the divider is configured to be melted. According to some embodiments, an actuator is adapted to selectively compromise the divider. According to some embodiments, a portion of at least one of the nitrite component and the acidic component is initially configured to be in solid form. According to some embodiments, a portion of at least one of the nitrite component and the acidic component is initially configured to be in liquid form. According to some embodiments, between about 200 nanomoles and about 600 nanomoles of nitric oxide is adapted to be delivered to the user per dose.

According to some embodiments, systems, methods and applications, a nitric oxide nasal delivery method comprises providing a container housing a nitrite component and an acidic component adapted to generate nitric oxide gas when combined. The nitrite component and the acidic component are configured to be separated by at least one divider during a storage period prior to generation of nitric oxide gas. The nitrite component and the acidic component are configured to be selectively combined by a user during a gas generation period to generate nitric oxide gas within the container. The selective combination is activated by the user selectively compromising the at least one divider separating the nitrite component and the acidic component, to enable mixing of the nitrite component and the acidic component within a reaction zone within the container, and to initiate the gas generation period. The method comprises providing a cap configured to be removably coupled to the container and adapted to seal the container during the storage period and during at least a portion of the gas generation period when coupled to the container. The cap is adapted to be removed from the container during a nasal delivery period when nitric oxide gas is adapted to be delivered from the reaction zone within the container, through an exit of the container. The method comprises providing a gas filter and/or membrane filter positioned between a reaction zone of the container and an exit of the container. The gas filter and/or membrane filter is configured to allow at least one dose of nitric oxide gas to pass through the gas filter and/or membrane filter and is configured to limit nitrogen dioxide gas from passing through the gas filter and/or membrane filter by substantially trapping nitrogen dioxide gas within the reaction zone of the container during the nasal delivery period. The method comprises providing a droplet filter and/or vapor filter positioned between the reaction zone of the container and the exit of the container; wherein the droplet filter and/or vapor filter is configured to allow at least one dose of nitric oxide gas to pass through the droplet filter and/or vapor filter and is configured to limit droplets from passing through the droplet filter and/or vapor filter by substantially trapping droplets within the reaction zone of the container during the nasal delivery period. The method comprises safely storing separately the nitrite component and the acidic component during the storage period. The method comprises selectively compromising the divider. The method comprises safely generating at least one dose of nitric oxide gas during the gas generation period. The method comprises safely delivering at least one dose of nitric oxide to the nasal cavity and/or mouth of the user during the nasal delivery period.

According to some embodiments, systems, methods and applications, the container comprises a body portion that is compressible. According to some methods, the nitrite component comprises one or more of potassium nitrite and sodium nitrite. According to some methods, the acidic component comprises one or more of hydrochloric acid, ascorbic acid, salicylic acid, and citric acid. According to some methods, the divider comprises at least one impermeable membrane. According to some methods, the divider is configured to be melted. According to some methods, an actuator selectively compromises the divider. According to some methods, a portion of at least one of the nitrite component and the acidic component is initially configured to be in solid form. According to some methods, at least one of the nitrite component and the acidic component is initially configured to be in liquid form. According to some methods, between about 350 nanomoles and about 450 nanomoles of nitric oxide is adapted to be delivered to the user per dose. According to some methods, between about 200 nanomoles and about 600 nanomoles of nitric oxide is adapted to be generated. According to some methods, between about 250 nanomoles and about 550 nanomoles of nitric oxide is adapted to be generated. According to some methods, between about 300 nanomoles and about 500 nanomoles of nitric oxide is adapted to be generated. According to some methods, between about 350 nanomoles and about 450 nanomoles of nitric oxide is adapted to be generated. According to some methods, between about 375 nanomoles and about 425 nanomoles of nitric oxide is adapted to be generated.

According to some embodiments, systems, methods, and applications, systems can comprise cartridges of reactants that can be positioned within reusable and/or disposable container and delivery systems as described in more detail herein.

### BRIEF DESCRIPTION OF THE DRAWINGS

Some embodiments of this disclosure will now be discussed in detail with reference to the following figures. These figures are provided for illustrative purposes only, and the embodiments are not limited to the subject matter illustrated in the figures.

FIG. 1 illustrates one embodiment of a nasal spray bottle system adapted to contain nitric oxide components and/or nitric oxide for nasal delivery according to some embodiments of the present disclosure.

FIG. 2 illustrates the system of FIG. 1 showing one or more internal components of a nasal spray bottle system according to some embodiments of the present disclosure.

FIG. 3 is a block diagram illustrating one embodiment of a method and/or protocol for using a single-use nasal spray device to deliver nitric oxide to a user's nasal cavity.

FIG. 4 is a block diagram illustrating one embodiment of a method and/or protocol for using a reusable nasal spray device to deliver nitric oxide to a user's nasal cavity.

FIG. 5 illustrates one embodiment of a nasal spray bottle system adapted to contain nitric oxide components and/or nitric oxide for nasal delivery according to some embodiments of the present disclosure showing one or more internal components of a nasal spray bottle system according to some embodiments of the present disclosure.

### DETAILED DESCRIPTION OF SOME PREFERRED EMBODIMENTS

Systems, products, compounds, and methods can be applied and/or used by a person to facilitate and promote the production of nitric oxide to be used by a person for health benefits. The systems, products, and devices include a nitric oxide nasal delivery system. The system can comprise one or more containers for compounds that when mixed are adapted to produce nitric oxide gas, at least one container with a nitrite compound and at least one container with an acid compound. Containers can be ruptured so that the compounds may be mixed and produce nitric oxide gas. The nitric oxide gas may then be delivered to a user via one or more of the nasal cavities and/or mouth.

The following detailed description is directed to certain specific embodiments. The invention(s) disclosed herein, however, can be embodied in a multitude of different ways as defined and covered by the claims. In this description, reference is made to the drawings, wherein like parts are designated with like numerals throughout. The features, aspects and advantages of the present invention will now be described with reference to the drawings of several embodiments that are intended to be within the scope of the development herein disclosed. These and other embodiments will become readily apparent to those skilled in the art from the following detailed description of the embodiments having reference to the attached figures, the invention not being limited to any particular embodiment(s) herein disclosed.

It will be readily understood that the components of the present invention, as generally described herein, could be arranged, and designed in a wide variety of different configurations or formulations. Thus, the following more detailed description of the embodiments of the systems, products, apparatus, and methods of the present invention, are not intended to limit the scope of the invention as claimed but are merely representative of various embodiments of the invention.

According to some preferred embodiments, the devices, systems, and methods described herein relate to a nitric oxide nasal spray. The nitric oxide nasal spray can be used to administer nitric oxide to the nasal cavity and/or mouth.

A user can obtain the benefits of nitric oxide therapy by utilizing a device designed to deliver nitric oxide gas to the nasal cavities or another passageway of the user. In some embodiments, the device includes a body that is compressible. In some embodiments, the device includes a nozzle that allows the nitric oxide nasal spray to exit the body of the device and enter the nasal cavity and/or passageway of the user. In some embodiments, the nozzle is preferably coupled to the body. The nozzle can be releasably coupled in some embodiments. The nozzle can be fixed to the body in some embodiments. In some embodiments, a cover and/or cap is included and adapted to cover the nozzle.

In some embodiments, a nitric oxide nasal dispenser comprises at least two containers, compartments, and/or separate cavities, within the body. In some embodiments, the containers, compartments, and/or separate cavities preferably comprise a breakable membrane and/or wall separating the contents of the containers, compartments, and/or separate cavities. According to some embodiments a device to facilitate rupturing the membrane and/or wall can be provided as described in more detail herein.

According to some embodiments, a first container preferably includes a first medium, such as a nitrite compound, sodium nitrite solution, and/or potassium nitrite, or the like. A second container preferably includes a second medium, such as an acidic compound, and/or acidic solution, or the like. For example, the acidic compound can include one or more of the following alone or in various combinations: hydrochloric acid, ascorbic acid, salicylic acid, and citric acid, etc. According to some embodiments, the use of ascorbic acid as the acidic reactant preferably promotes the production of pure nitric oxide, while limiting and/or avoiding production of nitrogen dioxide. According to some embodiments, the at least two containers preferably initially keep the respective compounds separate. In some embodiments, one or more of the containers can be ruptured in a manner that allows the respective compounds to mix or interact. Mixing and/or allowing the compounds to mix, initiates the production of nitric oxide gas in some preferred embodiments. In some embodiments, the nitric oxide gas may then be delivered to the user via the user's nasal cavity and/or passageway.

In some embodiments, the body of the device can contain a nitrite compound and/or medium and an acidic compound and/or medium. In some embodiments, the acidic compound can include one or more of the following, alone or in combination: hydrochloric acid, ascorbic acid, salicylic acid, lactic acid, and citric acid, etc. In some embodiments, the nitrite compound and/or medium and/or the acidic compound and/or medium are preferably dry components. For example, the dry components can be configured in the form of loose powder and/or a tablet that may be crushed and broken down to become loose powder. According to some embodiments, the body of the device includes a pouch. In some embodiments, the pouch can contain a liquid, such as, for example, water. Other liquids can also be used. In some embodiments, the pouch is adapted and configured to be ruptured in a manner that allows the respective compounds to mix and/or interact. Mixing and/or interacting can initiate the production of nitric oxide gas. In some embodiments, the nitric oxide gas can be delivered to the user via the user's nasal cavity and/or passageway.

According to some embodiments, a nasal spray system comprises a body and a nozzle. According to some embodiments, the body of the nasal spray system is filled with nitric oxide gas and sealed. In some embodiments, the nozzle is adapted to be coupled to the body. Actuating the nozzle can act to unseal the nitric oxide gas from the body and nitric oxide gas can be delivered through the nozzle to the nasal cavity. For example, according to some embodiments, a seal may be provided using one or more layers of a sealing material. According to some embodiments, a cap can be provided to the filled body to provide the seal. In some embodiments, a valve in the cap and/or nozzle preferably seals the nitric oxide gas within the body. In some embodiments, depressing the cap and/or nozzle and/or another component ruptures the seal to release nitric oxide through the nozzle. According to some embodiments the nitric oxide gas can be contained in a pressurized container with an appropriate pressure seal and/or valve. According to some embodiments, a pump can be provided to deliver the gas to the user via pumping action by a user or provider.

According to some embodiments, system, methods, and applications, the nasal applicator preferably is adapted and configured to deliver a suitable dose of nitric oxide to a user. For example, according to some applications a dose of nitric oxide is provided as a single dose, or as a series of multiple doses. According to some embodiments, nitrogen dioxide, to the extent present in the system, is preferably limited, minimized, filtered, avoided, and/or eliminated. According to some embodiments, an appropriately suitable dose of nitric oxide will contain a suitably low amount of nitrogen dioxide to maintain appropriately safe levels for the user.

According to some embodiments, a nitrite compound and/or medium can be formulated in a suitable manner and the concentration of reactants can be adapted to achieve the intended reaction and sufficient concentrations of nitric oxide. For example, according to some embodiments, ratios of the first medium may be formulated to include about 100 parts water (H₂O), about 10 parts sodium nitrite (NaNO₂), and about 2 parts baking soda (sodium bicarbonate, NaHCO₃). According to some preferred embodiments, ratios related to one or more amounts in their respective formulations may be adjusted within about 10%, within about 15%, within about 20%, within about 25%, within about 30%, within about 33%, and within about 50%. Furthermore, in some embodiments, additional ingredients may be added to one or more formulations for their respective media without disrupting or changing the efficacy of the medium.

According to some embodiments, an acidified compound and/or medium can be formulated in a suitable manner and the concentration of reactants can be adapted to achieve the intended reaction and sufficient concentrations of nitric oxide. For example, according to some embodiments, ratios of the second medium may be formulated to include about 100 parts water (H₂O), about 5 parts lactic acid (CH₃CH(OH)CO₂H), and about 8 parts citric acid (C₆H₈O₇). According to some preferred embodiments, ratios related to one or more amounts in their respective formulations may be adjusted within about 10%, within about 15%, within about 20%, within about 25%, within about 30%, within about 33%, and within about 50%. Furthermore, in some embodiments, additional ingredients may be added to one or more formulations for their respective media without disrupting or changing the efficacy of the medium. According to some embodiment, lactic acid and/or citric acid can be replaced with one or more of hydrochloric acid, ascorbic acid, salicylic acid, and/or citric acid.

In some embodiments, when approximately equal amount of the first medium and the second medium are combined, a reaction is initiated to produce nitric oxide gas. In some embodiments, the nitric oxide gas is then delivered to the user's nasal cavity through the user's nasal passage and/or mouth. According to some embodiments, a nozzle is used. According to some embodiments a cannula is used. According to some embodiments, a nasal mask is used. According to some embodiments, a face mask is used. In some embodiments, the nitric oxide gas is preferably absorbed by inhalation and/or via transdermal absorption.

In some embodiments, the body includes a reaction volume and/or space in which reactants are able to be mixed to initiate the production of nitric oxide. The reaction volume can include air, nitrogen, and/or other gas compounds. According to some embodiments, a first medium and a second medium are preferably held separately within the body of the device, until combined in some manner, which combination initiates the production of nitric oxide. In some embodiments, one or more reactants can be contained in one or more breakable or rupturable containers within the body. According to some embodiments, the first medium, in a liquid or solid state, can be in the body of the device and the second medium, in a liquid or solid state, can be added at the time intending to initiate the production of nitric oxide. According to some embodiments, squeezing the body causes the reactants to mix. According to some embodiments, pumping causes the reactants to mix. According to some embodiments, shaking the body causes the reactants to mix. According to some embodiments, placing a cap and/or nozzle to puncture a seal causes the reactants to mix. According to some embodiments, mechanically actuating a button and/or rupture tool can puncture a seal and causes the reactants to mix.

According to some embodiments, a nasal nitric oxide system comprises at least one or more filters or membranes. In some embodiments, a filter or membrane may be utilized to ensure that only gas exits the containment vessel and is inhaled by a user. In some embodiments, a filter or membrane can limit unwanted gas and other components from being inhaled and/or applied to the user during use. In some embodiments, more than one filter and/or membrane, of various suitable types, can be utilized. In some embodiments, the initiation of the production of nitric oxide can also produce nitrogen dioxide. In some embodiments, a suitable filter and/or membrane is used to filter out, minimize, and/or otherwise limit nitrogen dioxide gas. Accordingly, in some embodiments, it is preferred that nitric oxide gas exits the vessel and that nitrogen dioxide is impeded and limited from exiting the vessel during use. According to some embodiments and applications, the use of the at least one filter or/or membrane preferably helps to ensure that a suitable dose of nitric oxide gas is inhaled and/or absorbed by the user. According to some embodiments, nitrogen dioxide is predominantly retained in water vapor that is not administered with the dose of nitric oxide with the use of one or more filters and/or membranes. In some embodiments, filters can comprise plastic and/or other suitable filter materials and components as described further herein.

According to some embodiments, an appropriate amount of nitric oxide can be administered. In some embodiments, the reacted nitric oxide gas is able to pass through a filter system into the nasal cavity. In some embodiments, the dose administered can be an appropriate and suitable amount of nitric oxide. For example, according to one example assessment, without limitation, a relatively small amount of nitric oxide gas can be administered for an adult nasal application.

In some cases, an average maximum human adult breath can be approximately about six liters. In each of two nostrils approximately about three liters can be taken in during an average maximum human adult breath. According to some applications, for each nostril, an amount of nitric oxide of about 20 parts per million relative to about one half liter (0.5 L) approximate tidal volume can be a preferentially administered.

According to some embodiments, methods, and applications, a treatment dose of nitric oxide can preferably be about 400 nanomoles of nitric oxide. In some embodiments, a treatment dose of nitric oxide can preferably be between about 300 nanomoles and about 500 nanomoles of nitric oxide. According to some applications, a concentration of nitric oxide of about 20 parts per million in a breath can correspond to about 400 nanomoles, and/or between about 200 nanomoles and about 600 nanomoles. According to some embodiments and formulations, about 60 micro grams of sodium nitrite can react and correspond to produce about 400 nanomoles. According to some embodiments and formulations, about 160 micro grams of citric acid can react and correspond to produce about 400 nanomoles of nitric oxide in about a one-half liter breath. According to some embodiments and formulations, a suitable amount of water and/or solution can be used to dissolve and/or facilitate reaction of the reactants.

According to some embodiments, one or more suitable devices and/or materials Can be used to prevent and/or limit liquid droplets or mist from reaching a user's nasal cavities. In some embodiments, a filter or membrane may include a single layer of an interwoven material, such as a hydrophobic and/or acid resistant material. In some embodiments, the material used in a membrane or filter can depend on the type of filter used. For example, in some embodiments, a barrier filter may be used. The barrier filter can include a single layer of interwoven material which can be hydrophobic and acid resistant, such as Teflon or polypropylene, or the like. This may be a compact and light-weight filter. For example, filter materials of varying amounts of hydrophilicity can be used, such as, for example, polyvinyl difluoride (PVDF), polyester, and/or cotton. In some embodiments, a system can include a liquid filter, an antioxidant filter, and/or the like.

According to some embodiments, the delivery system can be disposable and/or designed to be of one-time use to produce and deliver a single dose of nitric oxide gas to transfer into the nasal cavity or passageway of the user in a safe manner. In some embodiments, a device can be constructed so that certain parts are reusable and certain parts are replaceable. For example, and not by way of limitation, in some embodiments, a pump, a nozzle, a reaction chamber, and/or a body of the device can be reusable, while a set of reaction packets and a set of filters may be replaceable. In some embodiments, used reaction packets and filters can be discarded after a single use and replaced with new reaction packets and filters. In some embodiments, reaction packets can be a single, durable cartridge pill or packet, which may be inserted and/or reinserted into a reaction chamber or body of the device. In some embodiments, reaction packets can be one or more cartridges, pills, tablets, pouches, and/or packets, that can be inserted into a reaction chamber or body of the device and discarded after use.

In some embodiments, one or more sets of reaction packets can include a first reaction packet that includes a nitrite compound and a second reaction packet that includes an acidic compound. The nitrite compound and the acidic compound can be in powdered form, or a suitable form preferably contained within the body of the device during use. In some embodiments, the nitrite compound can be potassium nitrite and/or sodium nitrite, or another suitable compound. In some embodiments, the acidic compound can be ascorbic acid, citric acid, a suitable food-grade acid, or another suitable acidic compound.

According to some embodiments, a user may separately crush the nitrite compound and the acidic compound with the user's hands or another suitable tool or mechanism prior to the nitrite compound and the acidic compound being contained within the body of the device. In some embodiments, at least a first reaction packet and at least a second reaction packet can be separated from each other by a fragile but impermeable wall and/or membrane that can remain inert and/or intact until intentionally broken by a force. For example, the impermeable wall or membrane can remain inert and/or intact until intentionally broken by the action of piercing, crushing, melting, or otherwise being opened and/or removed from between the first packet and the second packet in a combining action. In some embodiments, the intentional action may allow the resulting nitric oxide gas to fill the device prior to being expelled from the device in the form of an atomized spray consisting of nitric oxide gas and/or nitric oxide gas and a carrier liquid vapor. In some embodiments, a color indicator, or color change, may be used to signal that the production of nitric oxide has begun once the appropriate reactants have been mixed.

In some embodiments, the system and/or delivery device can include a battery-powered heating element. Other heating elements and or heat generators can also be used to facilitate and/or actuate mixing. The battery-powered heating element can be small and instantaneous. The body of the device can include a housing configured to house the battery-powered heating element. In some embodiments, the battery-powered heating element can be used to melt the wall, barrier, impermeable wall, and/or membrane that may be used to separate the first reaction packet and the second reaction packet and/or vaporize the first medium and the second medium. In some embodiments, a wall or membrane can be made of wax.

In some embodiments, the system and/or device includes an actuator, such as a plunger, a pin, a rod, and/or the like. The actuator can be positioned adjacent to the impermeable membrane or wall. In some embodiments, the system and/or device includes a moveable wall located within the body of the device that allows the actuator to move within the body of the device. For example, the actuator may move within the body of the device similar to a plunger within a syringe. In some embodiments, the actuator can be applied to cause the contents of the first packet and the second packet to interact by destroying the impermeable membrane or wall to initiate the intentional production of nitric oxide gas. The destruction of the impermeable wall or membrane, along with the mixing of the contents of the first and second packet, may occur simultaneously, or in a single compressive stroke, whereupon the actuator may spring back to its original position, rotating slightly in its travel in order to reset or re-register and ready itself for a secondary stroke, a series of strokes, and/or a final stroke, as needed and/or desired. Upon initiation, the body of the device may fill with the nitric oxide gas, which may be contained and ready for dispersion into the user's nasal cavity or passageway.

In some embodiments, nitric oxide gas is combined with purified water (distilled and/or deionized) and/or a sterile saline solution resulting in the formation of a vaporous mixture that can be expelled as nitric oxide mist into the nasal passageway or nasal cavity of the user.

In some embodiments, an actuator can be used to break a seal. For example, is some embodiments, re-registration of the actuator can bring the actuator into contact with a breakable seal within the moveable wall, which can cover an exit port of the device. In some embodiments, at the initiation of a second downward stroke, the actuator may tear or puncture the breakable seal. Additional compression may force the nitric oxide gas or vaporous mixture through the exit port of the device. In some embodiments, the system and/or device includes straightening screens preferably configured to provide a pre-determined discharge mist or vapor trail as the nitric oxide gas or vaporous mixture exits the device through a sterile mouth or nose piece. The mouth and/or nose piece can include an inlet airport to ensure proper vortex flow of the gas-air mixture into the nose and/or mouth of the user.

In some embodiments, the device and actuator can constitute a single-use device. In some embodiments, the device and actuator can be reusable and reloaded with additional first packets and second packets which can contain the first medium and the second medium. Such first packets and second packets can be sterile. Additionally, a sterile mouthpiece or nose guide can be provided for the device in a re-usable administration and can be installed prior to each use of the use.

In some embodiments, a method for administering nitric oxide via nasal spray through the use of a disposable device is provided. The method includes opening a package that can contain a device for single-use, disposable production of nitric oxide gas for delivery via a user's nasal passage or mouth. The device can include a body having a nozzle positioned at a top of the body, and at least one filter, a solution contained within the body, a pill contained within the body, and an impermeable membrane contained within the body separating the solution and the pill. The device can further include a threaded cap configured to cover a nozzle coupled to the body of the device. The solution can include sodium nitrite and purified water. The pill can include an acidic powder.

According to some applications, the method comprises pushing and/or squeezing the body of the device to crush the pill into a powder form. The method can include shaking the body of the device for approximately ten to fifteen seconds. The method can include removing the cap from the body and inserting the nozzle into a user's nasal cavity or passageway. The method can include squeezing the body of the device at least twice to force the delivery of nitric oxide gas to the user's nasal cavity.

In some embodiments, a method for administering nitric oxide via nasal spray through the use of a reusable device is provided. The method includes opening a package that can contain a reusable device that can deliver nitric oxide via nasal delivery, a gas filter and/or membrane filter packet containing a gas filter and/or membrane filter, a droplet filter and/or vapor filter packet containing a droplet filter and/or vapor filter, a first packet, and a second packet. The device can include a body, a nozzle, and a threaded cap. The first packet can include an acidic powder. The second packet can include a nitrite powder.

The method can include opening the nozzle and ensuring it is clean and unobstructed. The method can include opening the gas filter and/or membrane filter packet and inserting the gas filter and/or membrane filter into the nozzle, ensuring that it is seated well. The method can include opening the droplet filter and/or vapor filter packet and inserting the droplet filter and/or vapor filter into the nozzle, ensuring that it is seated well. The method can include adding clean water to the body of the device until the water reaches a fill line designated on the body of the device. The water can include tap water or deionized water. The method can include opening the first packet and emptying the first packing into the body of the device. The method can include opening the second packet and emptying the second packet into the body of the device.

The method can include threading the threaded cap to the body and shaking the body for approximately at least fifteen seconds to ensure proper mixing of the powdered components. The method can include removing the cap and inserting the nozzle into the user's nasal cavity. The method can include squeezing the body of the device at least twice. The method can include cleaning and/or removing the gas filter and/or membrane filter and the droplet filter and/or vapor filter for a future use.

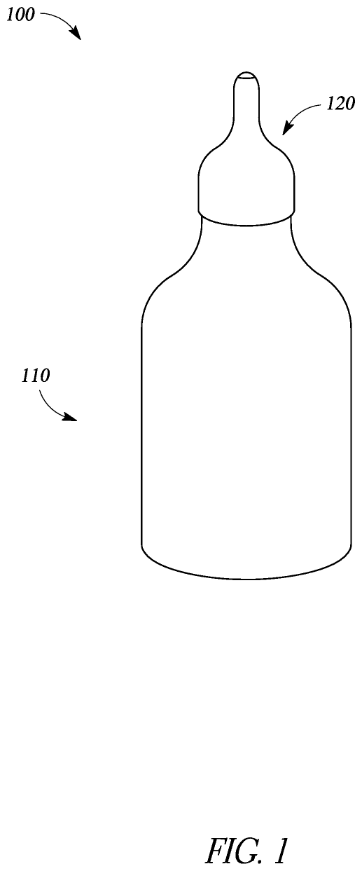

According to some embodiments, FIG. 1 illustrates a front view of an example system and embodiment of a nasal spray device 100. In some embodiments, the nasal spray device can include a body 110 and a cap 120. In some embodiments, the cap 120 can be a threaded cap. The body 110 can be made of a material that allows the body to be compressible and/or squeezable. The threaded cap 120 can attach to the body 110. Additionally, the threaded cap 120 can be removable from the body 110. In some other embodiments a cap can be fixed relative the body. In some embodiments, the cap is configured to seal the device. In some embodiments, the cap is configured to have an opening to direct the nasal spray into the nose of the user. In some embodiment, a portion of the cap can be ruptured during use to create a hole for delivery of the nitric oxide nasal spray.

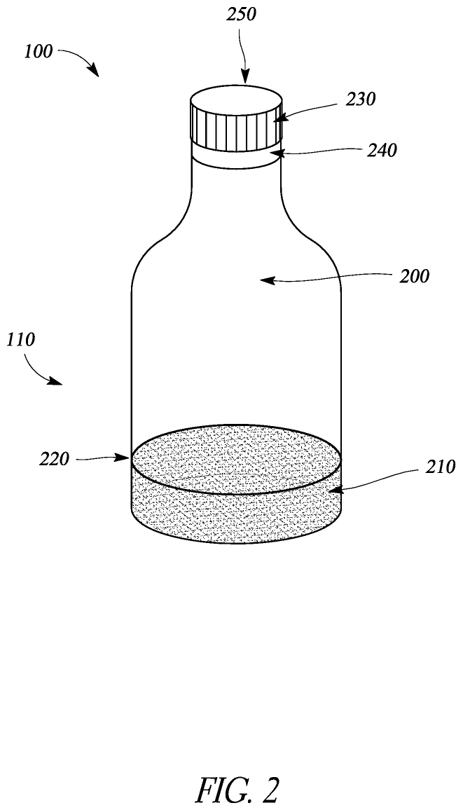

FIG. 2 illustrates a perspective schematic view showing internal components of the example system and embodiment that may or may not be visible depending on the opacity of the container and/or internal compartment 200 of the nasal spray device 100. In some embodiments, the body 110 of the device can be hollow. The internal compartment 200 of the body 110 can include a mixture 210. In some embodiments, the mixture 210 can include a nitrite compound and an acidic compound. For example, the nitrite compound can include potassium nitrite and/or sodium nitrite, or another suitable compound. For example, the acidic compound can include one or more of: hydrochloric acid, ascorbic acid, salicylic acid, lactic acid, and citric acid, etc. In some embodiments, the nitrite compound and the acidic compound can be separated by an impermeable wall or membrane. In some embodiments, the nitrite compound can be in a powdered form. In some embodiments, the acidic compound can be in a powdered form. In some embodiments, the acidic compound can be contained within a pill or tablet. In some embodiments, the mixture 210 can reach a fill line 220 as marked on the body 110 of the device.

In some embodiments, the device 100 can include a gas filter and/or membrane filter 230 and a droplet filter and/or vapor filter 240. The gas filter and/or membrane filter 230 can be used to filter out nitrogen dioxide that can be produced from the mixing of the nitrite compound and the acidic compound. The use of the gas filter and/or membrane filter can help to ensure that only an appropriate amount of nitric oxide gas is inhaled by the user. The use of the droplet filter and/or vapor filter can be used to help prevent liquid droplets or mist from reaching the user's nasal cavity or passageway. In some embodiments, the gas filter and/or membrane filter will be closest to the exit 250 of the body 110 of the device 100 and the droplet filter and/or vapor filter 240 can be positioned below the gas filter and/or membrane filter 230. In some embodiments, a pouch of water can be positioned inside the body 110 of the device 100.

In some embodiments a body can contain one or more pouches of components and/or reactants. For example, a pouch or container of an acidic component can be provided, such as a pouch with water and acid solution. In some embodiments, a pouch or container of nitrite reactant components can be provided. According to some applications one or more of the pouches can be ruptured and the contents mixed, by an actuator, and or by squeezing or otherwise puncturing the pouches or containers. In some embodiments the shape of the containers can be adjusted to improve mixing and/or facilitate stability during non-use/shipping and/or to facilitate rupture during intended application of the dose. According to some embodiments, a pouch or container can resemble the shape of a ball positioned within the container. A ball-shaped container can be easy to break in some embodiments. In some cases, a ball-shaped container can be passed through the neck of the device during manufacturing in a deflated condition. The deflated ball-shaped container can include a needle port, such that reactants can be delivered into the ball-shaped container while positioned within the body of the device using a syringe and needle to deliver the reactants within the ball-shaped container. The ball-shaped container can be filled and can increase in size to hold the reactants. The ball-shaped container can be ruptured during use or can otherwise have its contents expelled and mixed with the surrounding reactants. Collapsible and fillable containers of other shapes are also contemplated.

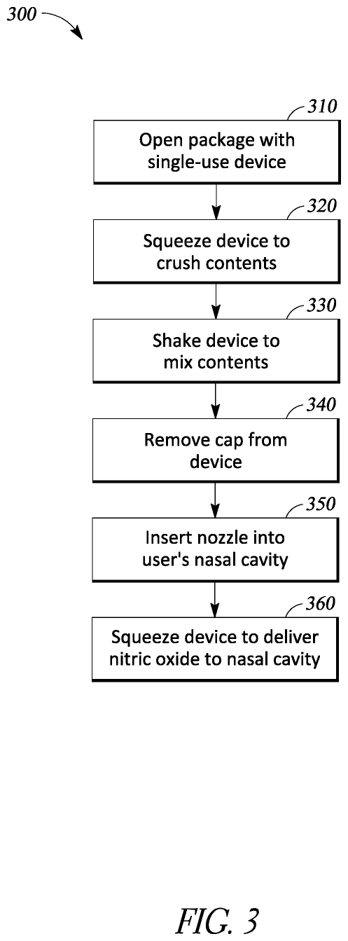

According to some embodiments and applications, FIG. 3 illustrates a method or protocol 300 for administration of a nasal spray device configured to deliver nitric oxide gas to a user. At block 310 the user can open a package that can contain a single-use device that can deliver nitric oxide via nasal delivery. In some embodiments, the device can include a body having a nozzle and at least one filter, a solution contained within the body, a pill contained within the body, and an impermeable membrane contained within the body separating the solution and the pill. In some embodiments, the device can further include a threaded cap configured to cover a nozzle coupled to the body. In some embodiments, the solution can include sodium nitrite and purified water. The pill can include an acidic powder.

At block 320, the user can squeeze the device to crush the internal components of the device. At block 330, the user can shake the device to mix the internal components of the device. In some embodiments, the shaking of the device can cause the internal components of the device to mix and produce nitric oxide gas. In some embodiments, the user can shake the device for approximately 15 seconds.

At block 340, the user can remove the cap from the device. At block 350, the user can insert the nozzle of the device into the user's nasal cavity or passageway. At block 360, the user can squeeze the device to deliver nitric oxide to the nasal cavity or passageway of the user. In some embodiments, the user can squeeze the device at least twice. According to some embodiments, a user can squeeze at least once in the left nostril and at least once in the right nostril.

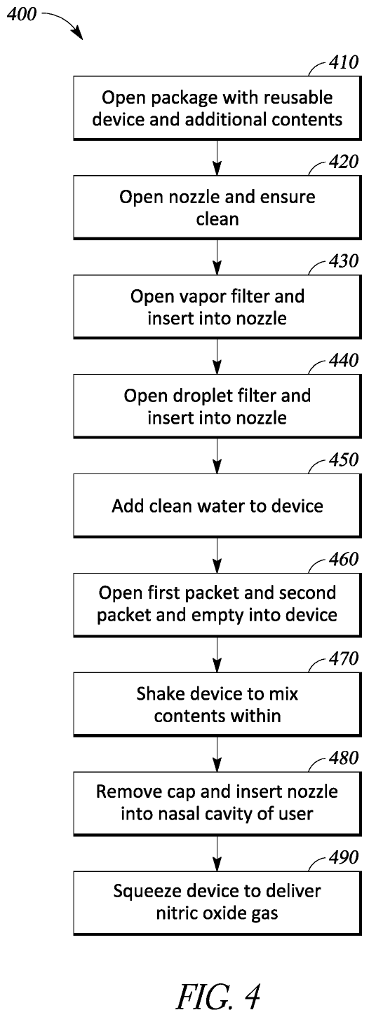

According to some embodiments and applications, FIG. 4 illustrates a method or protocol 400 for administration of a nasal spray device configured to delivery nitric oxide gas to a user. At block 410, the user can open a package that can contain a reusable device that can deliver nitric oxide via nasal delivery. In some embodiments, the package can further include a gas filter and/or membrane filter packet containing a gas filter and/or membrane filter, a droplet filter and/or vapor filter packet containing a droplet filter and/or vapor filter, a first packet, and a second packet. In some embodiments, the device can include a body, a nozzle, and a threaded cap. In some embodiments, the first packet can include an acidic powder. In some embodiments, the second packet can include a nitrite powder.

At block 420, the user can open the nozzle of the device and ensure that it is clean. At block 430, the user can open the gas filter and/or membrane filter and insert it into the nozzle of the device. At block 440, the user can open the droplet filter and/or vapor filter and insert it into the nozzle of the device.

In some embodiments, at block 450, the user can add clean water to the device. At block 460, the user can open the first packet and empty it into the device. In some embodiments, the user can also open the second packet and empty it into the device.

At block 470, the user can shake the device to mix the contents within the device. In some embodiments, this can produce nitric oxide gas. At block 480, the user can remove the cap of the device and insert the nozzle into the nasal cavity or passageway of the user. At block 490, the user can squeeze the device to deliver nitric oxide gas to the nasal cavity or passageway. In some embodiments, the user can squeeze the device at least twice. In some embodiments the nozzle can include a pump. The pump can deliver a dose of therapeutic gas through the filter system to the user by depressing the nozzle toward the bottle in some embodiments.

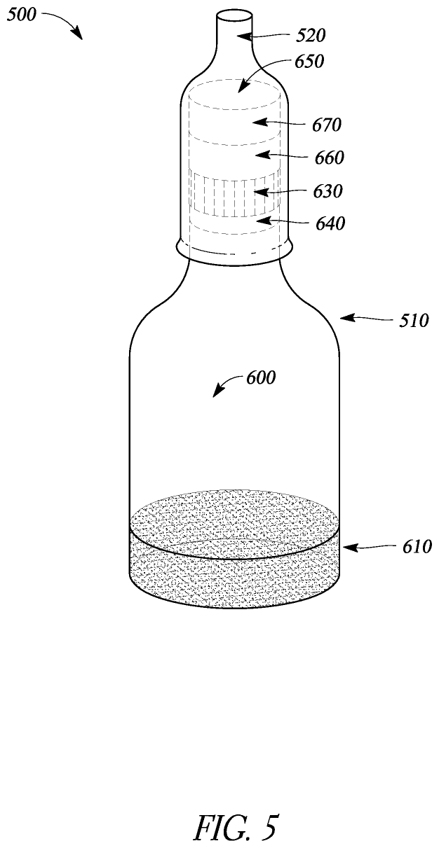

According to some embodiments, FIG. 5 illustrates a perspective schematic view showing internal components according to some systems and devices for nasal delivery of nitric oxide. In some embodiments, the nasal spray device 500 can include a body 510 and a cap 520. In some embodiments, the cap 520 can be a threaded cap. The body 510 can be made of a material that allows the body to be compressible and/or squeezable. The threaded cap 520 can attach to the body 510. Additionally, the threaded cap 520 can be removable from the body 110. In some other embodiments a cap can be fixed relative the body. Internal components of the example system and embodiment that may or may not be visible depending on the opacity of the container and/or internal compartment 600 of the nasal spray device 500. In some embodiments, the body 510 of the device can be hollow. The internal compartment 600 of the body 510 can include a mixture 610. In some embodiments, the mixture 610 can include a nitrite compound and an acidic compound. According to some embodiments, for example, the nitrite component and portion can include one or more of the nitrite ingredients, mediums and/or packet/containers disclosed herein. According to some embodiments, for example, the acidic component and portion can include one or more of the acidic ingredients, mediums and/or packet/containers disclosed herein. In some embodiments, the nitrite compound and the acidic compound can be separated by an impermeable wall or membrane or separate packet structures, including fillable and/or deflatable packet structures. In some embodiments, the nitrite compound can be in a powdered form or a solution form. In some embodiments, the acidic compound can be in a powdered form or a solution form. In some embodiments, the nitrite compound can be contained within a packet, pouch, pill or tablet. In some embodiments, the acidic compound can be contained within a packet, pouch, pill or tablet. In some embodiments, the mixture 610 can reach a fill line marked on the body 510 of the device.

In some embodiments, the device 500 can include multiple filters, barrier layers, pumps, nozzels, and/or caps. The filters, layers, caps, can be in various suitable orders. For example, a gas filter and/or membrane filter 630 and a droplet filter and/or vapor filter 640. The gas filter and/or membrane filter 630 can be used to filter out nitrogen dioxide that can be produced from the mixing of the nitrite compound and the acidic compound. The use of the gas filter and/or membrane filter can help to ensure that only an appropriate amount of nitric oxide gas is inhaled by the user. The use of the droplet filter and/or vapor filter 640 can be used to help prevent liquid droplets or mist from reaching the user's nasal cavity or passageway. In some embodiments, one or more gas filter and/or membrane filters can be nearer to the exit 650 of the body 510 of the device 500, and the droplet filter and/or vapor filter 640 can be positioned below the gas filter and/or membrane filter 630. In some embodiments, a pouch of water and/or solution can be positioned inside the body 510 of the device 500. Additional advantageous features and components described herein can be configured, incorporated, used, and/or adapted to be included within a nasal nitric oxide delivery system as disclosed herein.

While the above detailed description has shown, described, and pointed out novel features of the development as applied to various embodiments, it will be understood that various omissions, substitutions, and changes in the form and details of the devices illustrated can be made by those skilled in the art without departing from the spirit of the development. As will be recognized, the present development can be embodied within a form that does not provide all the features and benefits set forth herein, as some features can be used or practiced separately from others. All changes which come within the meaning and range of equivalency of the claims are to be embraced within their scope. Additional disclosure and relevant information supporting this application is also provided in the following patents and applications, each of which is hereby incorporated by reference herein in its entirety: U.S. Pat. Nos. 7,939,045; 9,138,707; 10,517,817; 8,685,467; 10,052,348; 8,720,436; 8,434,475; 9,649,467; 8,501,090; and U.S. patent application Ser. Nos. 17/727,651, 17/727,630, 17/494,180, are each incorporated by reference herein and made part of this disclosure.

The foregoing description details some embodiments of the systems, devices, and methods disclosed herein. It will be appreciated, however, that no matter how detailed the foregoing appears in text, the systems, devices, and methods can be practiced in many ways. As is also stated above, it should be noted that the use of particular terminology when describing certain features or aspects of the invention should not be taken to imply that the terminology is being re-defined herein to be restricted to including any specific characteristics of the features or aspects of the technology with which that terminology is associated.

It will be appreciated by those skilled in the art that various modifications and changes can be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the Figures can be combined, interchanged, or excluded from other embodiments.

With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “comprising” as used herein is synonymous with “including,” “containing,” or “characterized by,” and is inclusive or open-ended and does not exclude additional, unrecited elements or method steps.

The above description discloses several methods of manufacture and materials of the present development. This development is susceptible to modifications in the methods and materials, as well as alterations in the fabrication methods and equipment. Such modifications will become apparent to those skilled in the art from a consideration of this disclosure or practice of the development disclosed herein. Consequently, it is not intended that this development be limited to the specific embodiments disclosed herein, but that it covers all modifications and alternatives coming within the true scope and spirit of the development as embodied in the attached claims.

While the above detailed description has shown, described, and pointed out novel features of the improvements as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all the features and benefits set forth herein, as some features may be used or practiced separately from others. The scope of the invention is indicated by the appended claims rather than by the foregoing description. All changes which come within the meaning and range of equivalency of the claims are to be embraced within their scope.

What is claimed is:

1. A nitric oxide nasal delivery system, comprising:
a container housing a nitrite component and an acidic component adapted to generate nitric oxide gas when combined, wherein the nitrite component and the acidic component are configured to be separated by at least one divider during a storage period prior to generation of nitric oxide gas, wherein the nitrite component and the acidic component are configured to be selectively combined by a user during a gas generation period to generate nitric oxide gas within the container, wherein the selective combination is activated by the user selectively compromising the at least one divider separating the nitrite component and the acidic component, to enable mixing of the nitrite component and the acidic component within a reaction zone within the container, and to initiate the gas generation period;
a cap configured to be removably coupled to the container and adapted to seal the container during the storage period and during at least a portion of the gas generation period when coupled to the container, and adapted to be removed from the container during a nasal delivery period when nitric oxide gas is adapted to be delivered from the reaction zone within the container, through an exit of the container;
a membrane filter positioned between a reaction zone of the container and an exit of the container; wherein the membrane filter is configured to allow at least one dose of nitric oxide gas to pass through the membrane filter and is configured to limit nitrogen dioxide gas from passing through the membrane filter by substantially trapping nitrogen dioxide gas within the reaction zone of the container during the nasal delivery period; and
a droplet filter positioned between the reaction zone of the container and the exit of the container; wherein the droplet filter is configured to allow at least one dose of nitric oxide gas to pass through the droplet filter and is configured to limit droplets from passing through the droplet filter by substantially trapping droplets within the reaction zone of the container during the nasal delivery period;
wherein the system is adapted to safely store separately the nitrite component and the acidic component during the storage period, safely generate at least one dose of nitric oxide gas during the gas generation period after the divider is selectively compromised by the user, and safely deliver at least one dose of nitric oxide to the nasal cavity and/or mouth of the user during the nasal delivery period.

2. The system of claim 1, wherein the container comprises a body portion that is compressible.

3. The system of claim 1, wherein the nitrite component comprises one or more of potassium nitrite and sodium nitrite.

4. The system of claim 1, wherein the acidic component comprises one or more of hydrochloric acid and citric acid.

5. The system of claim 1, wherein the divider comprises at least one impermeable membrane.

6. The system of claim 1, wherein the divider is configured to be melted.

7. The system of claim 1, comprising an actuator adapted to selectively compromise the divider.

8. The system of claim 1, wherein a portion of at least one of the nitrite component and the acidic component is initially configured to be in solid form.

9. The system of claim 1, wherein a portion of at least one of the nitrite component and the acidic component is initially configured to be in liquid form.

10. The system of claim 1, wherein between about 200 nanomoles and about 600 nanomoles of nitric oxide is adapted to be delivered to the user per dose.

11. A nitric oxide nasal delivery method, comprising:
providing a container housing a nitrite component and an acidic component adapted to generate nitric oxide gas when combined, wherein the nitrite component and the acidic component are configured to be separated by at least one divider during a storage period prior to generation of nitric oxide gas, wherein the nitrite component and the acidic component are configured to be selectively combined by a user during a gas generation period to generate nitric oxide gas within the container, wherein the selective combination is activated by the user selectively compromising the at least one divider separating the nitrite component and the acidic component, to enable mixing of the nitrite component and the acidic component within a reaction zone within the container, and to initiate the gas generation period;
providing a cap configured to be removably coupled to the container and adapted to seal the container during the storage period and during at least a portion of the gas generation period when coupled to the container, and adapted to be removed from the container during a nasal delivery period when nitric oxide gas is adapted to be delivered from the reaction zone within the container, through an exit of the container;
providing a membrane filter positioned between a reaction zone of the container and an exit of the container; wherein the membrane filter is configured to allow at least one dose of nitric oxide gas to pass through the membrane filter and is configured to limit nitrogen dioxide gas from passing through the membrane filter by substantially trapping nitrogen dioxide gas within the reaction zone of the container during the nasal delivery period; and
providing a droplet filter positioned between the reaction zone of the container and the exit of the container; wherein the droplet filter is configured to allow at least one dose of nitric oxide gas to pass through the droplet filter and is configured to limit droplets from passing through the droplet filter by substantially trapping droplets within the reaction zone of the container during the nasal delivery period;
safely storing separately the nitrite component and the acidic component during the storage period;
selectively compromising the divider;
safely generating at least one dose of nitric oxide gas during the gas generation period; and
safely delivering at least one dose of nitric oxide to the nasal cavity and/or mouth of the user during the nasal delivery period.

12. The method of claim 11, wherein the container comprises a body portion that is compressible.

13. The method of claim 11, wherein the nitrite component comprises one or more of potassium nitrite and sodium nitrite.

14. The method of claim 11, wherein the acidic component comprises one or more of hydrochloric acid and citric acid.

15. The method of claim 11, wherein the divider comprises at least one impermeable membrane.

16. The method of claim 11, wherein the divider is configured to be melted.

17. The method of claim 11, comprising an actuator adapted to selectively compromise the divider.

18. The method of claim 11, wherein a portion of at least one of the nitrite component and the acidic component is initially configured to be in solid form.

19. The method of claim 11, wherein a portion of at least one of the nitrite component and the acidic component is initially configured to be in liquid form.

20. The method of claim 11, wherein between about 300 nanomoles and about 500 nanomoles of nitric oxide is adapted to be generated.
